(19) United States
(12) Patent Application Publication
(10) Pub. No.: US 2006/0003384 A1
(43) Pub. Date: Jan. 5, 2006

(54) Detection of measurement of antibodies to antigenic proteins in biological tissues or samples

(76) Inventors: Carrie L. Wagner, Malvern, PA (US); Robert Jordan, Thornton, PA (US); Jeannie Rojas, Doylestown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/170,311

(22) Filed: Jun. 29, 2005

Related U.S. Application Data
(60) Provisional application No. 60/584,374, filed on Jun. 30, 2004.

(51) Int. Cl. G01N 33/53

(52) U.S. Cl. 435/7.1

(57) ABSTRACT

The present invention relates to methods and compositions for detecting and/or measuring serum antibodies to antigenic proteins in a sample, comprising adding a labeled antigenic protein or fragment thereof to a sample derived from serum and expected to contain serum antibodies and measuring differences in at least one characteristic between (a) a labeled serum antibody-antigenic protein complex; (b) an serum antibody-antigenic protein complex in the sample; and/or (c) displaced lableled or unlabeled serum antibody, antigenic protein or fragment thereof.

This application is based on U.S. 60/584,374, filed Jun. 30, 2004, which is entirely incorporated here by reference.

### FIELD OF THE INVENTION

The present invention in the field of biotechnology and medical diagnostics, relates to methods for detecting and/or measuring therapeutic or induced antibodies to antigenic proteins in a sample, comprising (a) adding a labeled or unlabeled antigenic protein or fragment thereof to a sample expected to contain therapeutic or induced antibodies, and (b) measuring differences in at least one characteristic between (i) a labeled antibody-antigenic protein complex; (ii) an unlabeled antibody-antigenic protein complex in the sample; and/or (iii) displaced labeled or unlabeled antibody, antigenic protein or fragment thereof.

### BACKGROUND OF THE INVENTION

In response to the presence of foreign or mis-recognized endogenous proteins, the body can produce antibodies, termed herein as “induced antibodies” which include antibodies to antigenic proteins or therapeutic proteins, such as therapeutic antibodies, or antibodies to endogenous proteins that are involved, e.g., in inflammatory, infectious, autoimmune, aging, or neurological diseases or pathologies and related conditions. Meanwhile, a therapeutic antibody may form an immune complex with its target. One important aspect of such medical treatment is to detect the presence and/or measure the amount of induced antibodies or immune complexes in a patient's immune response to such therapy or autoimmune condition.

Such detection or measurement is important as a tool in the diagnosis and/or evaluation of treatment parameters to determine which and how much therapeutic protein, antibody or other treatment should be used. For example, if a patient is given a therapeutic protein for treatment and the patient subsequently produces induced antibodies against the therapeutic protein, the amount of induced antibodies in the serum could be determined to find out how to modify the dosage or type of therapeutic protein administered. Alternatively, the presence and amount of induced antibodies to endogenous proteins in an autoimmune patient can be evaluated to diagnose and/or determine appropriate treatment for particular diseases and pre-pathological or pathological conditions.

Prior methods have utilized known immunoassay methods to attempt to measure induced antibody responses to particular therapeutic or endogenous proteins. However, these methods have been unreliable. One problem associated with the methods involves using reagent antibodies to detect and distinguish induced antibodies from other non-immune antibodies. In addition, there are challenges in specifically detecting complexes consisting of the induced antibody and the antigenic protein, e.g., where the complexes are not clearly distinguishable from the uncomplexed induced antibody or other non-immune antibodies present in patient serum. These difficulties have made previous methods less useful in diagnosis or evaluation of treatment of pathological conditions or effects associated with biologic therapies.

Accordingly, there is a need to provide alternative methods for detecting and/or measuring therapeutic or induced antibodies to antigenic proteins that are suitable for diagnosis or evaluation of treatment in patients having autoimmune conditions or conditions that can be treated using therapeutic proteins.

### SUMMARY OF INVENTION

The present invention provides at least one method for the detection and/or measurement of induced antibodies to antigenic proteins. Such antigenic proteins or fragments thereof can include endogenous, foreign or administered proteins, such as, but not limited to, antibodies or fragments, such as therapeutic antibodies, therapeutic proteins, genetically engineered proteins and labeled or derivatized proteins.

The present invention provides a new method utilizing at least one detectably labeled or unlabeled antigenic protein or fragment thereof, where the detectable label can include, inter alia, at least one radiolabel and/or at least one other suitable marker, or any combination thereof. Such method of the present invention can include, but is not limited to, the use of characteristic differences (e.g., size, physical or chemical characteristic, and/or label differences) between (1) the induced antibody-antigenic protein complex; (2) the labeled induced antibody-antigenic protein complex; and/or (3) displaced components thereof, to detect or measure induced antibody in biological samples, e.g., but not limited to, serum, plasma, whole blood, cerebrospinal fluid (CSF), lymph or tissue homogenates,

In a preferred embodiment of the present invention, radiolabeled and/or detectably labeled antigenic protein or fragments thereof can be used to displace unlabeled antigenic protein from the induced antibody-antigenic protein complex. The labeled induced antibody-antigenic protein complex can then be distinguished and/or resolved from the unlabeled protein complex, and/or free unlabeled antigenic protein by different retention times using chromatography or other methods (e.g., HPLC size exclusion chromatography), indicating changed molecular weight. Since human serum contains many serum proteins, it can be difficult to distinguish the labeled induced antibody-antigenic protein complex from other high molecular weight endogenous components in the serum via UV absorbance, dynamic light scattering or other known methods. The labeled induced antibody-antigenic protein complex is detected based on molecular size, label, tag, amplification of the label or tag, and/or the ability of the labeled antigenic protein to bind to at least one detectable substrate.

### DESCRIPTION OF THE DRAWINGS

FIG. 1 shows a counts per minute (CPM) chromatogram of a radiolabeled antigenic protein that has been resolved by size on an HPLC column and detected via the radiolabel.

FIG. 2 shows a CPM chromatogram of an immune complex of an antigenic protein and a radiolabeled monoclonal antibody to the antigenic protein that has been resolved by size on an HPLC column and detected via the radiolabel.

FIG. 3 shows a CPM chromatogram of an immune complex of an antigenic protein and a monoclonal antibody to the antigenic protein which has been incubated in the presence of excess radiolabeled antigenic protein. The profile of these proteins is shown following separation by size on an HPLC column and detection via the radiolabel.

FIG. 4 shows a CPM chromatogram of an immune complex of an antigenic protein and a monoclonal antibody to the antigenic protein, which has been incubated in the presence of excess radiolabeled non-immune IgG.

FIG. 5 shows a CPM chromatogram of an immune complex of an antigenic protein and a polyclonal antibody to the antigenic protein, which has been incubated in the presence of excess radiolabeled antigenic protein. The profile of these proteins is shown following separation by size on an HPLC column and detection via the radiolabel.

FIG. 6A is a CPM chromatogram of baseline patient serum sample taken prior to the initiation of an infliximab (anti-TNF antibody) treatment regimen. FIG. 6B is a CPM chromatogram of patient serum taken from the same patient 28 weeks after the initiation of the treatment (8 weeks after the latest infliximab infusion). Each sample was incubated with radiolabeled infliximab followed by separation on an HPLC column and detected via the radiolabel.

FIG. 7 shows a CPM chromatogram of serum taken from a patient 62 weeks after the initiation of the treatment (8 weeks after the latest infliximab infusion). The sample was incubated with radiolabeled infliximab followed by separation on an HPLC column and detected via the radiolabel.

FIG. 8 shows a CPM chromatogram of serum taken from a patient 110 weeks after the initiation of the treatment (more than 8 weeks after the latest infliximab infusion). The sample was incubated with radiolabeled infliximab followed by separation on an HPLC column and detected via the radiolabel.

FIG. 9 is a graphical representation showing PCR amplification of an anti-biotin antibody-DNA conjugate bound to biotinylated infliximab.

FIG. 10 shows an expanded CPM chromatogram of an induced antibody-antigenic protein complex. The antigenic protein is infliximab. The complex is shown in the presence or absence of infliximab Fab (iFab) and detected via the radiolabel.

FIG. 11 shows an expanded CPM chromatogram of serum taken from a patient positive with induced antibody against infliximab. The sample was first incubated with unlabeled infliximab and then with 125I-labeled infliximab Fab fragment (125I-iFab). It was separated on an HPLC column and detected via the radiolabel.

### DETAILED DESCRIPTION

The present invention in the field of biotechnology and medical diagnostics, relates to methods for detecting and/or measuring therapeutic or induced antibodies to antigenic proteins in a sample, comprising (a) adding a labeled or unlabeled antigenic protein or fragment thereof to a sample expected to contain therapeutic or induced antibodies, and (b) measuring differences in at least one characteristic between (i) a labeled antibody-antigenic protein complex; (ii) an unlabeled antibody-antigenic protein complex in the sample; and/or (iii) displaced labeled or unlabeled antibody, antigenic protein or fragment thereof.

Citations

All publications or patents cited herein are entirely incorporated herein by reference as they show the state of the art at the time of the present invention and/or to provide description and enablement of the present invention. Publications refer to scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2005); Sambrook, et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2005); Colligan et al., Current Protocols in Protein Science, John Wiley & Sons, NY, N.Y., (1997-2005). Furuya, D., et al., Journal of Immunological methods 238 (2000): 173-180.

The antigenic proteins include, for example, therapeutic proteins, diagnostic proteins, antibodies, natural or genetically engineered proteins, protein complexes, labeled and derivatized proteins, peptides, and peptide mimetic. The proteins and related molecules can be either endogenous or foreign to the animal or human. The present invention also applies to antigenic substances such as small molecules, nucleic acids, carbohydrates, and lipids. The antigenic substances can be involved in therapy and diagnosis of, for example, therapeutic antibody treatable diseases, autoimmune, neurological and other diseases, aging and the like. The present invention applies to sample types including, but not limited to sera, plasma, isolated blood cells, lymph, CSF, tissues, tissue homogenates, and the like, as well known in the art.

The characteristics that can be measured in the present invention include, but not limited to, retention time, molecular weight, buoyant density, fluorescence polarization, poly-ethylene glycol (PEG) precipitation, and/or those known in the art. The labels that can be used include, but not limited to, radiolabels (I123, I125, C14, H3, etc.), DNA labels, nucleic acid labels, fluorescent labels, enzymatic labels, chemiluminescence or other labels. The labeled displaced antibody amounts may be quantitatively correlated with the type, amount and affinity of the induced antibody. Labeled or unlabeled proteins and complexes can be separated by chromatography (HPLC, TLC, etc.), mass spectroscopy, ultracentrifugation, sucrose density gradient ultracentrifugation, analytical ultracentrifugation, electrophoresis, and/or other methods known in the field. See, e.g., Ausubel, Harlow and Lane and Colligan, et al., supra, and the like, which are entirely incorporated herein by reference.

According to the present invention, antibody titer may be determined by any method known to the art using standard techniques, including, but not limited to, ELISA, RIA, EIA, and other solid phase immunoassays, radioimmunoassay, nephelometry, rocket electrophoresis, Western blot, immunofluorescence, cell based assays, etc. See, e.g., Ausubel, Harlow and Lane and Colligan, et al., supra, and the like, which are entirely incorporated herein by reference.

In the following non-limiting examples, samples were analyzed using an Integral HPLC Workstation (Applied Biosystems, Foster City, Calif.) configured in the single column mode with a BioSep 3000 size exclusion column (Phenononex, Torrance, Calif.) and detected using an ABI dual UV detector at 280 and 220 nm followed by a radioactivity detector (Packard Instrument Company, Downers Grove, Ill.). These techniques are by way of example only, and the invention can include any known method, technique, or material, as well known in the art, based on the teaching and guidance presented herein.

### EXAMPLE 1

### Detection of Experimentally Formed Antigen and Monoclonal Antibody Immune Complex by Intercolation of Labeled Antigen into the Immune Complex

The immune complex of antigenic protein (infliximab, 15.3 ug/mL) and induced murine monoclonal antibody to the antigenic protein (5.1 ug/mL, 3:1 molar ratio) was experimentally formed in normal human serum. At these specified concentrations, the induced monoclonal antibody was completely bound by the excess antigenic protein and not detectable using current in vitro assay formats.

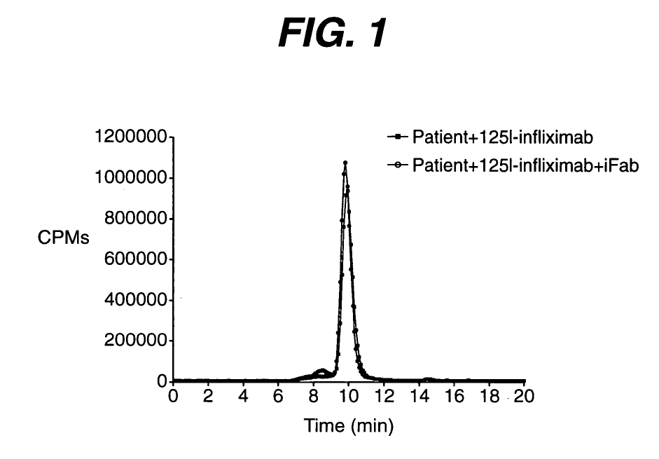

The CPM chromatogram in FIG. 1 shows that the retention time of 125I-labeled antigenic protein (infliximab) is approximately 16.4 minutes, which is characteristic of the protein's size and shape. The retention time remains relatively constant when the HPLC column, flow parameters and mobile phase buffer, are left unchanged.

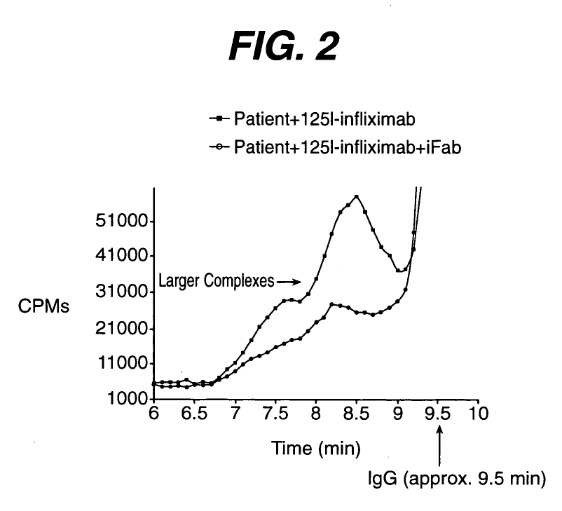

The immune complex of an antigenic protein and its induced antibody is larger in size than each of the individual component. Accordingly, its retention time should be shorter than that of the uncomplexed antigenic protein or induced antibody. As shown in FIG. 2, the retention time of the immune complex of infliximab (15.3 ug/mL) and the radiolabeled induced monoclonal antibody against infliximab (5.1 ug/mL) is approximately 14.8 minutes. This is shorter than 16.4 minutes, the retention time of the 125I-labeled infliximab (FIG. 1).

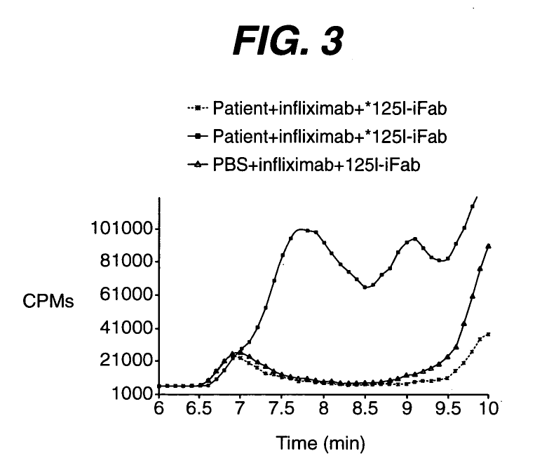

To demonstrate that induced monoclonal antibody can be detected through radiolabeled antigenic protein, serum containing immune complex of infliximab and induced monoclonal antibody against infliximab was incubated in the presence of excess 125I-labeled infliximab for 1 hour at 37 degrees. FIG. 3 shows the CPM chromatogram with peaks at 24.8, 16.8 and 14.8 minutes. These are retention times characteristic of free 125I not associated with protein (24.8 minutes), uncomplexed 125I-labeled infliximab (16.8 minutes), and immune complex of 125I-labeled infliximab and induced murine antibody (14.8 minutes), respectively. It indicates that a portion of 125I-labeled infliximab was able to integrate into the unlabeled preformed immune complex (retention time at 14.8 minutes), while the excess labeled antigenic protein remained unbound (retention time at 16.8 minutes). Therefore, induced murine antibody to infliximab was detected via the ability of excess 125I-labeled infliximab to displace unlabeled infliximab in the existing immune complex.

As a control, serum containing immune complex of infliximab and induced monoclonal antibody against infliximab was incubated in the presence of excess 125I-labeled normal non-immune monkey IgG for 1 hour at 37 degrees. The normal non-immune monkey IgG is non-specific for either component of the preformed immune complex, i.e., it is not capable of binding to either infliximab or the induced monoclonal antibody. Its retention time is the same as that of infliximab (which is also an IgG1 antibody). FIG. 4 shows the CPM chromatogram with a single peak at 16.4 minutes, which the retention time characteristic of 125I-labeled normal non-immune monkey IgG. It indicates that non-specific 125I-labeled protein is not able to integrate into the unlabeled preformed immune complex.

### EXAMPLE 2

### Detection of Experimentally Formed Antigen and Polyclonal Antibody Immune Complex by Intercolation of Labeled Antigen into the Immune Complex

The immune complex of antigenic protein (infliximab, 15.3 ug/mL) and induced monkey polyclonal antibody to the antigenic protein (5.1 ug/mL, 3:1 molar ratio) was experimentally formed in normal human serum. At these specified concentrations, the induced polyclonal antibody was completely bound by the excess antigenic protein and not detectable using current in vitro assay formats.

To demonstrate that induced polyclonal antibody can be detected through radiolabeled antigenic protein, serum containing immune complex of infliximab and induced polyclonal antibody against infliximab was incubated in the presence of excess 125I-labeled infliximab for 1 hour at 37 degrees. FIG. 5 shows the CPM chromatogram with peaks at 24.8, 16.8, 14.4 and 13.2 minutes. These are retention times characteristic of free 125I not associated with protein (24.8 minutes), uncomplexed 125I-labeled infliximab (16.8 minutes), and complexes with variable sizes and stoichiometry of 125I-labeled infliximab and induced polyclonal antibody (14.4 and 13.2 minutes), respectively. It indicates that a portion of 125I-labeled infliximab was able to integrate into the unlabeled preformed immune complex (retention time at 14.4 and 13.2 minutes), while the excess labeled infliximab remained unbound (retention time at 16.8 minutes). Therefore, induced polyclonal antibodis to infliximab were detected via the ability of excess 125I-labeled infliximab to displace unlabeled infliximab in the existing immune complex.

### EXAMPLE 3

### Detection of Infliximab and Induced Anti-Infliximab Antibody Immune Complexes in Patient Serum

Serum samples were taken from patient A at week 0 and week 28 after the initiation of infliximab treatment (8 weeks after the latest infliximab infusion). Both were determined by double antigen EIA analysis to be negative for induced antibodies to infliximab. No circulating infliximab was detectable using a validated ELISA in either sample.

The serum was incubated with approximately 15 μg/mL of ¹²⁵I-labeled infliximab for at least one hour at 37 degrees on a shaking platform. For serum sample taken at week 0 (FIG. 6A), a single peak was detected at 16.4 minutes, the retention time of uncomplexed ¹²⁵I-labeled infliximab. There is no significantly visible peak at less than 16.4 minutes (the percentage of the area under the chromatogram of retention time less than 16.4 minutes over the total chromatogram area is approximately 11.6%), which suggests that no complex with higher molecular weight is present. Similar pattern was observed for serum sample taken at week 28 (FIG. 6B) with a single peak at 16.4 minutes, and the area under the chromatogram of retention time less than 16.4 minutes represents approximately 14.9% of the total chromatogram area. Therefore, the HPLC analysis confirms the absence of an induced immune response.

In another experiment, serum samples were taken from patient B at week 62 after the initiation of infliximab treatment (8 weeks after the latest infliximab infusion). It was determined by double antigen EIA analysis to be negative for induced antibodies to infliximab. However, circulating infliximab was not detectable using a validated ELISA in this sample. Accordingly, this serum sample is considered inconclusive, i.e., no detectable induced antibody, but circulating antigenic protein (infliximab) is present.

The serum was incubated with approximately 15 μg/mL of ¹²⁵I-labeled infliximab for at least one hour at 37 degrees. The CPM chromatogram (FIG. 7) shows a single peak was detected at 16.4 minutes and no significantly visible peak at less than 16.4 minutes, which suggests that no complex with higher molecular weight is present. This pattern is similar to those in FIGS. 6A and 6B, in which the sera were known to be negative for antibodies to infliximab by ELISA. Therefore, the HPLC analysis shows that the serum sample which was inconclusive based on ELISA is negative of an induced immune response.

In another experiment, serum samples were taken from patient C at week 110 after the initiation of infliximab treatment (more than 8 weeks after the latest infliximab infusion). It was determined by double antigen EIA analysis to be positive for induced antibodies to infliximab (titer 1:10).

The patient serum was incubated with approximately 15 μg/mL of ¹²⁵I-labeled infliximab for at least 1 hour at 37 degrees. The CPM chromatogram (FIG. 8) shows peaks at retention times of 16.4, 14.0 and 11.6 minutes. The retention times of 14.0 and 11.6 minutes are indicative of immune complexes of ¹²⁵I-labeled infliximab and induced antibodies against infliximab. Therefore, the HPLC analysis confirms the presence of an induced immune response.

### EXAMPLE 4

### Immuno-PCR Amplification System

A non-radioactive, immuno-PCR system was developed to detect the presence of induced antibody. In this assay format, if the immune complex is present in serum sample, biotinylated infliximab, which displaces the unlabeled infliximab in the complex, can be detected using an anti-biotin antibody-DNA conjugate, followed by PCR amplification of the conjugates DNA label.

Serial dilutions of biotinylated infliximab were coated onto NUNC polycarbonate immuno-PCR wells. The plate was then blocked with nonfat dried milk in buffer containing salmon sperm DNA to block nonspecific DNA binding. The blocked plate was probed for biotinylated infliximab using a mouse anti-biotin antibody conjugated to a 5′-amidated 227 base pair, double-stranded DNA molecule. After extensive washing, internal primers and PCR reagents were added directly to the wells and the plate was subjected to PCR amplification. A biotinylated infliximab dose dependent specific amplification was shown in FIG. 9. The detection limit was around 450 fg (1.7×10⁶ molecules) biotinylated infliximab.

### EXAMPLE 5

### Detection of Induced Anti-Infliximab Antibodies Using ¹²⁵I-Labeled Infliximab or ¹²⁵I-Labeled Fab Fragment of Infliximab

In this example, patient serum was determined by a double antigen EIA analysis to be positive for induced antibodies to infliximab. However, no free infliximab was detectable in this sample.

In one experiment, the serum was incubated with 70 μg/mL of ¹²⁵I-labeled infliximab at 37 degrees for at least 1 hour to form ¹²⁵I-labeled infliximab-induced antibody complexes. The sample was reanalyzed in the double antigen EIA and was rendered to inconclusive due to the absence of signal. An excess of infliximab Fab fragment (iFab) was added to the preformed immune complex and incubated for at least 1 hour at 37 degrees. The sample was separated and counted on an HPLC system. Fractions (0.25 mL) were collected using a Gilson fraction collector and aliquots were then counted using a Topcount Microscintillation Counter. The retention time of ¹²⁵I-labeled infliximab (molecular weight=149 kD) was approximately 10 minutes, consistent for a human Ig on this HPLC system. The ¹²⁵I-labeled infliximab-induced antibody complex resolved as a smaller series of peaks that eluted between 7 to 9 minutes (FIG. 10). Following incubation with iFab, the height of the immune complex peak was reduced, indicating that iFab displaced some of the ¹²⁵I-labeled infliximab in the immune complex. This suggests that antigenic protein-induced antibody complex can be detected through fragment (iFab) displacement of labeled antigenic protein (infliximab).

In another experiment, the serum was incubated with an excess of unlabeled infliximab at 37 degrees F. for at least 1 hour to form unlabeled infliximab-induced antibody complexes. An excess of ¹²⁵I-labeled iFab was added to the preformed immune complex and incubated for at least 1 hour at 37 degrees F. The sample was separated and counted on an HPLC system. The retention time for ¹²⁵I-iFab is approximately 11.3 minutes. As shown in FIG. 11, following the addition of ¹²⁵I-iFab, distinctive peaks were observed in the 7 to 9 minute region, indicating that ¹²⁵I-iFab was incorporated into the unlabeled complexes. This suggests that antigenic protein-induced antibody complex can be detected using labeled protein antigenic fragment (iFab).

It will be clear that the invention can be practiced otherwise than as particularly described in the foregoing description and examples. Numerous modifications and variations of the present invention are possible in light of the above teachings and, therefore, are within the scope of the claims.

What is claimed is:

1. A method for detecting and/or measuring induced antibodies to antigenic proteins in a biological sample, comprising
(a) Adding a labeled or unlabeled antigenic protein or a labeled or unlabeled fragment thereof to a sample containing said induced antibodies; and
(b) Measuring differences in at least one characteristic between displaced and non-displaced induced antibody or antigenic protein in at least one of:
(i) Labeled induced antibody-antigenic protein complex;
(ii) Unlabeled induced antibody-antigenic protein complex; and/or
(iii) Displaced labeled or unlabeled induced antibody, antigenic protein or fragment thereof.

2. A method according to claim 1, wherein said characteristic is at least one selected from: molecular weight, size exclusion, chromatography retention time, detectable label, amplifiable label, and buoyant density.

3. A method according to claim 1, wherein said protein is at least one selected from at least one peptide, protein, glycoprotein, lipoprotein, antibody, antibody fragment, fusion protein, genetically engineered protein, and protein conjugate.

4. A method according to claim 3, wherein said protein is at least one selected from an exogenous protein and an endogenous protein.

5. A method according to claim 4, wherein said exogenous protein is selected from a therapeutic protein, an antigenic protein, a protein of an infectious organism and a diagnostic protein.

6. A method according to claim 5, wherein said therapeutic or diagnostic protein is an antibody or fragment thereof.

7. A method according to claim 6, wherein said antibody is specific for an endogenous protein.

8. A method according to claim 7, wherein said endogenous protein is at least one selected from insulin, EPO, TPA, FSH, PTH, at least one growth hormone, cytokine, receptor, autoantigen, transcription factor or fragment thereof.

9. A method according to claim 8, wherein said cytokine is selected from IL-1, IL-2, IL-3, IL-4, IL-10, IL-12, IL-13, IL-14, IL-18, IL-21, IL-23, IL-27, tumor necrosis factor, CD-3 or tissue factor.

10. A method according to claim 8, wherein said receptor or fragment is selected from TNF receptor, GPIIbIIIa, alphaVbeta1, 2, 3, 4, 5, 6, 7, or 8.

11. A method according to claim 6, wherein said antibody is specific for at least one exogenous protein.

12. A method according to claim 11, wherein said exogenous protein is at least one selected from a protein of an infectious agent.

13. A method according to claim 12, wherein said infectious agent is at least one selected from a bacteria, a virus, a mold, a fungus, a prion.

14. A method according to claim 4, wherein said endogenous protein is selected from an autoantigenic protein, a pathologic protein
